(19) United States
(12) Patent Application Publication
(10) Pub. No.: US 2014/0171850 A1
(43) Pub. Date: Jun. 19, 2014

(54) HEAT AND ANESTHETIC PACK

(71) Applicant: LESLIE HUNTER, New Braunfels, TX (US)

(72) Inventor: LESLIE HUNTER, New Braunfels, TX (US)

(21) Appl. No.: 13/718,456

(22) Filed: Dec. 18, 2012

(51) Int. Cl. A61M 35/00

(52) U.S. Cl. CPC A61M 35/00
USPC 602/48

(57) ABSTRACT

A heat and anesthetic pack comprising an instant heat pack, where the instant heat pack includes a front side and a back side; an occlusive dressing attached to a central portion of the front side of the instant heat pack; a central blister pack attached to a center portion of the occlusive dressing, where the central blister pack includes a numbing cream; and a peel-away covering positioned above the occlusive dressing and the central blister pack, where the peel-away covering remains in place until a user is ready to use. During use the user places the heat and anesthetic pack on the user's skin with the central blister pack and the occlusive dressing exposed to the skin. The numbing cream numbs the skin while the heat pack promotes drainage.

### CROSS REFERENCE TO OTHER APPLICATIONS

This application claims priority to U.S. Provisional Application Ser. No. ______ filed on Dec. 18, 2011.

### BACKGROUND OF THE INVENTION

1. Field of the Invention

The present invention relates to a kit that provides noninvasive anesthetic techniques to prepare a patient for minor surgical procedures.

2. Description of Related Art

Traditionally, a patient in need of surgery requires anesthesia by means of an injection to numb the affected area. Less invasive methods like creams and even ice may be employed to treat the area, but they are not as effective as the subcutaneous injection. Injections are especially worrisome for individuals who fear needles, causing them anxiety before having to undergo a minor surgical procedure. The creams offer minor relief but are typically not as effective because they dry up and lose their effectiveness after prolonged exposure to air.

Another method of pain management is heat. Heat therapy or thermotherapy is when heat is applied to manage pain, especially useful for arthritis and stiff muscles. The application of heat increases blood flow thereby providing more nutrients carried in the blood. Thermotherapy manages some pain, but alone is still not as effective as an injection for isolated minor pain relief. Therefore, it would be beneficial in the art to combine the methods of topical creams with heat application to manage pain for patients before minor surgical procedures.

### SUMMARY OF THE INVENTION

In view of the foregoing disadvantages inherent in the prior art, the general purpose of the present invention is to provide a skin pack that combines heat and topical cream, configured to include all of the advantages of the prior art, and to overcome the drawbacks inherent therein.

Accordingly, an object of the present invention is to provide a heat and anesthetic pack which includes an instant heat pack and a blister pack filled with a numbing cream, where the heat and anesthetic pack is applied to a patient's skin before minor surgery.

Another object of the present invention is to provide a heat and anesthetic pack that includes an occlusive dressing to keep the heat pack and blister pack in place while numbing the patient's skin.

To achieve the above objects, in an aspect of the present invention, a heat and anesthetic pack is described comprising an instant heat pack, where the instant heat pack includes a front side and a back side; an occlusive dressing attached to a central portion of the front side of the instant heat pack; a central blister pack attached to a center portion of the occlusive dressing, where the central blister pack includes a numbing cream; and a peel-away covering positioned above the occlusive dressing and the central blister pack, where the peel-away covering remains in place until a user is ready to use. During use the user places the heat and anesthetic pack on the user's skin with the central blister pack and the occlusive dressing exposed to the skin. The numbing cream numbs the skin while the heat pack promotes drainage.

These together with other aspects of the present invention, along with the various features of novelty that characterize the present invention, are pointed out with particularity in the claims annexed hereto and form a part of this present invention. For a better understanding of the present invention, its operating advantages, and the specific objects attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description and claims taken in conjunction with the accompanying drawings in which:

FIG. 1 depicts a perspective view of a heat and anesthetic pack in accordance with an exemplary embodiment of the present invention.

### DETAILED DESCRIPTION OF THE DRAWINGS

The present invention relates to a kit that provides noninvasive anesthetic techniques to prepare a patient for minor surgical procedures. The present invention provides a heat and anesthetic pack to manage pain prior to minor surgery. The heat and anesthetic pack incorporates a disposable heat pack and an occlusive clear dressing infused with an accurate dose of 4% lidocaine cream. The heat and anesthetic pack is intended for use at least 15 minutes before the surgical procedure to allow the lidocaine cream to adequately numb the intended area of skin. The heat pack provides added comfort for patients requiring minor procedures like abscess draining, lumber punctures, and peripheral intravenous line insertion.

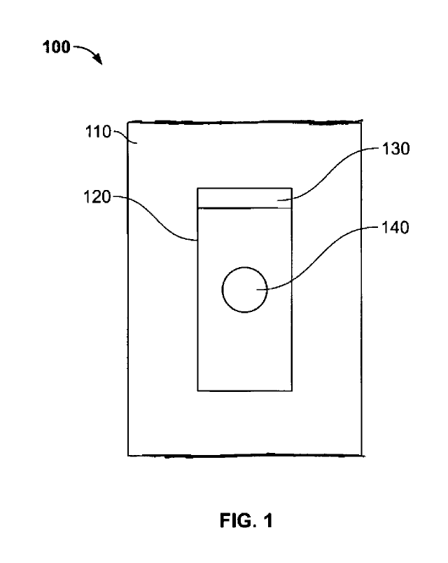

Turning now descriptively to the drawing, FIG. 1 shows a perspective view of a heat and anesthetic pack 100 in accordance with an exemplary embodiment of the present invention. The heat and anesthetic pack 100 includes an instant heat pack 110, an occlusive dressing 120, a central blister pack 140, and a peel-away covering with tab 130. The heat and anesthetic pack 100 may be available in various sizes to accommodate skin wounds in a range of sizes. The heat and anesthetic pack 100 may be shaped as a rectangle with preferred dimensions of 12 cm by 6 cm. Other shapes like circles, squares, or triangles may be used, but the preferred shape is a rectangle.

The occlusive dressing 120 is air and water tight for sterile wound coverage. To use, the peel-away covering with tab 130 is removed from the occlusive dressing 120 to expose the sticky side of the occlusive dressing 120 and the central blister pack 140. The central blister pack 140 contains 4% lidocaine cream which upon exposure to the user's skin starts to numb the top skin layers. After about 15 minutes the skin is numb and ready for the surgical procedure. The instant heat pack 110 is composed as a bandage to soften the skin, increase blood flow and allow abscesses to drain efficiently, therefore eliminating the need for incision and drainage.

All of the components of the heat and anesthetic pack 100 are layered one on top of the other to create the entire pack. The instant heat pack 110 is the first layer, followed by the occlusive dressing 120 attached to the front side of the instant heat pack 110. Then the central blister pack 140 is centrally positioned on the occlusive dressing 120 with the peel-away covering with tab 130 covering both the occlusive dressing 120 and the central blister pack 140. The central blister pack 140 is closest to the user's skin to numb the skin while the instant heat pack 110 remains the outermost layer to comfortably increase blood flow and promote effective draining The backside of the instant heat pack 110 is exposed to the air while the front side (illustrated in FIG. 1) is in contact with the user's skin.

Before placing the heat and anesthetic pack 100 the instant heat pack 110 must be activated by squeezing the edges of the pack 110 together and then kneading it to activate the inner heating mechanisms. Next the user pulls the tab of the peel-away covering 130 to expose the central blister pack 140 and the occlusive dressing 120. The user then places the front side of the instant heat pack 110 upon the desired area of the user's skin to begin the numbing and heating process. After 15 min the heat and anesthetic pack 100 may be removed to begin the minor surgical procedure.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A heat and anesthetic pack comprising:
a. an instant heat pack, where the instant heat pack includes a front side and a back side;
b. an occlusive dressing attached to a central portion of the front side of the instant heat pack;
c. a central blister pack attached to a center portion of the occlusive dressing, where the central blister pack includes a numbing means; and
d. a peel-away covering positioned above the occlusive dressing and the central blister pack, where the peel-away covering remains in place until a user is ready to use the heat and anesthetic pack.

2. The heat and anesthetic pack according to claim 1, where the occlusive dressing includes a sticky layer to attach the occlusive dressing to a patient's skin.

3. The heat and anesthetic pack according to claim 1, where the occlusive dressing is air and water-tight.

4. The heat and anesthetic pack according to claim 1, where the peel-away covering includes a tab to pull off the covering.

5. The heat and anesthetic pack according to claim 1, where the central blister pack includes 4% lidocaine cream to serve as the numbing means.

6. A method of use for a heat and anesthetic pack comprising the steps of:
a. activating an instant heat pack;
b. peeling away a covering on the instant heat pack, where the covering exposes an occlusive dressing and a central blister pack;
c. positioning the central blister pack and the occlusive dressing on a user's skin; and
d. numbing the user's skin with a numbing cream within the central blister pack.

7. The method of use for the heat and anesthetic pack according to claim 6 further comprising the step of waiting 15 minutes before removing the heat and anesthetic pack to begin a minor surgical procedure.
